(19) United States
(12) Patent Application Publication
(10) Pub. No.: US 2026/0213024 A1
(43) Pub. Date: Jul. 23, 2026

(54) METHODS FOR DETERMINING INTERDEPENDENT ATTRIBUTES OF COMMUNICATIONS IN A DIGITAL COMMUNICATION NETWORK

(71) Applicant: Sequelae, Inc., Minneapolis, MN (US)

(72) Inventors: Duncan Hieu Minh Dam, Palm Springs, CA (US); Sara Taylor, Edina, MN (US); Alex Ruch, Prosper, TX (US); Salomea Anna Jankovic, Minneapolis, MN (US)

(73) Assignee: Sequelae, Inc., Minneapolis, MN (US)

(21) Appl. No.: 19/450,249

(22) Filed: Jan. 15, 2026

Related U.S. Application Data
(60) Provisional application No. 63/748,148, filed on Jan. 22, 2025.

(51) Int. Cl. G16H 70/60

(52) U.S. Cl. CPC G16H 70/60

(57) ABSTRACT

Methods and systems to determine interdependent attributes in communications of a digital communication network (DCN) and using the interdependent attributes to promote positive communications to slow a progression of an adverse health condition are provided. The method provides a DCN for members of a population to communicate with each other. A plurality of messages from members of the population are selected to be shown to the individual member. The DCN analyzes the messages to determine a first degree to which the communication contains an idea is useful in slowing a progression of an adverse health condition and a second degree to which the idea in the communication will spread in the DCN. The DCN presents the plurality of messages to the individual member in the prioritized order.

### CROSS REFERENCE TO RELATED APPLICATIONS

This application claims the benefit of U.S. Provisional Application No. 63/748,148 filed on Jan. 22, 2025, which application is incorporated herein by reference in its entirety.

### BACKGROUND

Various embodiments relate generally to health care systems, methods, devices and computer programs and, more specifically, relate to determining interdependent attributes of communications in a digital communication network and using the interdependent attributes of the communications to slow a progression of an adverse health condition.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Digital communication networks, which can include social media where individuals can interact with others online, connect many with a community. For example, a digital communication network can be used to connect members using the same health insurance or health care. Computers can also interact with people using sophisticated programs such as, for example, chat bots. These programs can mimic human interaction and provide information to people. The online interactions, whether through a community or a computer bot, can be utilized to help build positive behaviors and encourage people to make improvements in their lives.

A digital network can also provide information to its users through messages and other interactions. The digital network can prioritize messages and present opportunities to users which can improve their engagement. However, it may be difficult to determine such prioritization without knowing the motivation behind communications from a user and the interdependent attributes of the communications. For example, knowing whether a user is communicating within the digital network to manage a disease the user has or to manage weight loss may be useful in determining the prioritization of messages to send to the user. In such example, if the user is seeking disease management, communications that are directed to or motivated by disease management from other users may be prioritized.

Further, in some conventional digital networks, messages are prioritized to get members to engage with the network as frequently as possible in order to increase engagement with the digital network. This may result in prioritizing messages that induce tribalism, anger, and/or fear in members.

Thus, what is needed is a way to prioritize communications that are directed to and helpful in promoting a healthy lifestyle through positive engagement for a user or member in a digital communication network.

### SUMMARY

Example aspects of the present disclosure include:

A method according to at least one embodiment of the present disclosure comprises providing a digital communication network (DCN) for a plurality of members of the population to communicate with each other; analyzing a plurality of communications on the DCN, the plurality of communications generated by the plurality of members; determining, based on the analysis of the plurality of communications, a first degree to which each communication contains an idea that is useful in slowing a progression of an adverse health condition and a second degree to which an idea will spread in the DCN; prioritizing an order in which the individual member sees the plurality of communications based on at least the first degree to which the communication contains the idea that is useful in slowing the progression of the adverse health condition that the member has and the second degree to which the idea will spread in the DCN; and presenting the plurality of communications to the individual member in the order, wherein messages with higher levels of prioritization are shown first.

Any of the aspects herein, wherein the idea includes at least one of an action and an experience undertaken by the member who generated the communication, and wherein the idea includes a motivation of the action by the member who generated the communication.

Any of the aspects herein, wherein the action comprises at least one of: lifestyle changes, medical interventions, changes in social interactions, and changes in a living environment.

Any of the aspects herein, wherein the idea is associated with an ontology created automatically based on the analysis of the plurality of communications.

Any of the aspects herein, wherein the adverse health condition is associated with an ontology created automatically based on the analysis of the plurality of communications.

Any of the aspects herein, further comprising: generating, using a language model, a target communication to the individual member based on the first degree and the second degree; and presenting the target communication to the individual member.

Any of the aspects herein, wherein the target communication comprises at least one of a recommendation for the individual member directed to improving an adverse health condition of the individual member or a summary of the plurality of communications.

Any of the aspects herein, wherein the individual member has the adverse health condition.

Any of the aspects herein, wherein the method is used to slow the progression of the adverse health condition of the individual member.

Any of the aspects herein, wherein the adverse health condition is determined by automatically analyzing communications from the individual member in the DCN.

Any of the aspects herein, wherein the adverse health condition comprises at least one of chronic systemic inflammation, malaise, low energy, a disease, a health risk, social dysfunction, or a prodromal disease.

Any of the aspects herein, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

Any of the aspects herein, wherein the health risk is at least one of falling or becoming infected with an illness.

A method according to at least one embodiment of the present disclosure comprises providing a digital communication network (DCN) for a plurality of members of the population to communicate with each other; analyzing a plurality of communications on the DCN, the plurality of communications generated by the plurality of members; determining, based on the analysis of the plurality of communications, a motivation for each communication of the plurality of communications that contains an idea useful in slowing a progression of an adverse health condition; prioritizing an order in which the individual member sees the plurality of communications based on the motivation of each communication of the plurality of communications; and presenting the plurality of communications to the individual member in the order, wherein communications with motivations matching a motivation of the individual member are prioritized higher.

Any of the aspects herein, wherein the idea includes at least one of an action and an experience undertaken by the member who generated the communication, and wherein the idea includes a motivation of the action by the member who generated the communication.

Any of the aspects herein, wherein the action comprises at least one of: lifestyle changes, medical interventions, changes in social interactions, and changes in a living environment.

Any of the aspects herein, wherein the idea is associated with an ontology created automatically based on the analysis of the plurality of communications.

Any of the aspects herein, wherein the adverse health condition comprises at least one of chronic systemic inflammation, malaise, low energy, a disease, a health risk, social dysfunction, or a prodromal disease.

Any of the aspects herein, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

A system according to at least one embodiment of the present disclosure comprises a computer processor; a data repository in communication with the computer processor and storing: communications, a first degree, and a second degree; a digital communications network (DCN) which, when executed by the computer processor, provides a network for a plurality of members of a population to interact with at least each other or the DCN; and a server controller which, when executed by the computer processor: analyzes communications on the DCN, the communications generated by the plurality of members; determines, based on the analysis of the communications, a first degree to which each communication contains an idea that is useful in slowing a progression of an adverse health condition and a second degree to which an idea will spread in the DCN; prioritizes an order in which the individual member sees the communications based on at least the first degree to which the communication contains the idea that is useful in slowing the progression of the adverse health condition that the member has and the second degree to which the idea will spread in the DCN; and presenting the communications to the individual member in the order, wherein messages with higher levels of prioritization are shown first.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4A shows an example of a computing system, in accordance with one or more embodiments.

FIG. 4B shows an example of a network, in accordance with one or more embodiments.

### DETAILED DESCRIPTION

Digital communications networks, especially the subset known as social networks, are typically financed through a business model based on advertising. As previously described, they focus on getting members to engage with the network as frequently and deeply as possible in order to draw more members into their fold. They do this through prioritizing messages in a members communication feed designed to maintain engagement. As anyone who has experienced one of the modern social networks can see, this is often done by playing into tribalism, anger and/or fear.

Various embodiments use a digital communication network (DCN) to promote a healthy lifestyle. The various embodiments can also be used to manage a disease, lower a health risk in a population, manage health care costs, and increase or improve the effectiveness of communications in the DCN. The DCN is intended to promote positive engagement as opposed to conventional DCN's that rely on negative engagement. Such positive engagement is to encourage members to take positive actions to improve their health. These improvements can be lifestyle changes, adoption and compliance with medical interventions, activities that induce a positive emotional state, and the like. Members both give and receive the benefits of ideas and support through communication on the DCN. Through this process the members build self-efficacy and agency, the belief that they can impact their own outcomes through their actions, and this agency extends to other aspects of their lives beyond their health, increasing overall wellbeing.

Like a conventional system, the DCN can be optimize for engagement. However, rather than focus on engagement alone, without consideration of the repercussions of the potential negative emotions, the DCN may instead spread “action” and generate agency messages which have certain attributes. Four such attributes are empathy, attachment, introspection, and self-efficacy. The DCN can derive these attributes from analysis of messages, and optimizes for action out of the network in part by prioritizing messages that have the type of attributes which drive this outcome.

Empathy, attachment, introspection, and agency are attributes of communications that can contribute to the efficacy of a DCN in spreading ideas as medicine. They are different from (and in some ways at odds with) standard engagement which is driven by advertising-based models. As with other attributes, the DCN may use artificial intelligence (AI) bots trained on sample data, such as test results, in order to assign a ranking for the attribute to a message. The DCN may also use such AI bots to generate messages which intended to reflect (or induce) such attributes, for example, by being ranked highly in one or more such attributes.

The DCN can analyze individual messages to gauge the level of the communication attributes. Alternatively, the DCN may analyze multiple messages in a conversation in order to assign an associated level of the communication attributes to the conversation (either in part or in whole). The DCN can also evaluate conversations over multiple periods of time in order detect changes in the communication attributes, for example, to compare the level of agency from a first month to the level of agency from a second, subsequent month.

The DCN can also analyze one or more messages to determine an interdependency of attributes between messages. Such interdependent attribute may be, for example, motivation behind various communications, a degree to which a communication contains an idea that is useful to a user, and/or a degree to which an idea will spread in the DCN. More specifically, the one or more messages may be analyzed to determine a degree to which the message contains an idea that is useful to a user (such as, for example, slowing the progression of an adverse health condition) and a degree to which an idea will spread in the DCN. The idea can include an action or experience undertaken by the user who sent the message and a motivation behind the action or the experience. The motivation may be, for example, introspection of the user's health and actions for improving their health. Such information can be useful in matching or prioritizing messages for a user with similar or matching motivations. For example, a user may be interacting with the DCN to obtain more information or ideas for disease management. Messages that can be helpful in the disease management and that are also motivated by disease management can be prioritized over messages that are motivated by other reasons such as social interaction.

The generated messages may be used to communicate information regarding various lifestyle options, such as interventions, health-inducing tools available to the population, opportunities to engage in reflection, etc. Some generated message can provide the knowledge directly, direct the user to a tool to help them achieve healthier lifestyles and/or communicate information regarding the various tools available.

The individual messages may be relayed separately, for example, in an ordered sequence. The order messages are provided may be arranged based on the rankings for various attributes, such as those messages having higher empathy rankings being shown before those with lower empathy rankings. Likewise, some message may be omitted if the desired attribute ranking does not reach a given threshold.

The DCN may also generate summaries of the multiple communications. Similar types of communications may be combined and analyzed to create the summaries. The summaries may include copies of the communications, directions to access the communications and/or snippets of the communications. The order of the communications in the summaries may be based on the rankings of the attributes for each communication.

Additionally, the user's engagement can be used to provide the user's experience as a social aspect. A social value can be placed on those members of a community that have relevant experience. Further, the user's motivation can be social. For example, a member may wish to connect and talk with other members experiencing a similar or the same type of chronic pain. Further, the engagement from the users can be induced in order to help elevate the aspects of the experience that may be more instructional to the community as a whole. This can help increase the number of members of the community thinking about an action, for example, creating an atmosphere where people can learn about others in who have undergone a change they are considering. Users who have positive engagement with a message may be well situated to create wide-range engagement in the community.

Additionally, the community can be used to help support the patient in other ways. Lifestyle interventions are inherently safe. They use the philosophy that any action now is preferred to a “better” action later. They also support the concept that ideas and communication are healthcare.

Medical interventions, such as pharmaceuticals, can be used over the long-term to mitigate the consequence, severity, or discomfort associated with a disease. In most cases this is the intended use, at least until the user's underlying disease progresses to the state where an even more powerful intervention may be recommended. This can often be the case even when the disease has a basis in the user's lifestyle. Patients and manufacturers look at the drug as a replacement to making lifestyle changes which can be harder to adopt. Finding and adopting lifestyle changes that work is a cognitively and emotionally challenging process that is incompatible for most people while with dealing the underlying disease and its symptom at the same time.

Interventions can be used in ways to allow short-term use of the intervention to give the patient cognitive and emotional room to better appreciate that lifestyle change is possible (and preferred over a lifetime of pharmaceutical use). However, people can seize upon the benefits of the intervention if they take the time to consider their conditions more fully. Various embodiments are directed to incentivize user engagement using a digital communication network (DCN).

Positive engagement can help a user associate with an intervention and foster an environment that allows the intervention to be used as a tool rather than a crutch. Enabling a user to earn incentives for lifestyle promoting activities, and then use those incentives to obtain other therapies can also help incentivize the user to engage in those activities. Additionally, embodiments may be used to slowing progression of an adverse health condition or to manage a disease.

Lifestyle change has traditionally been looked at as in individual pursuit, such as plans personalized just for the patient. Given its connection to the health system this is not surprising. Medicine is a one-on-one activity (reinforced by the privacy concepts the system is based on). Unfortunately, lifestyle change is highly driven by social parameters and the impact on social parameters is critical (but often ignored by the health care system).

Communities provide support, ideas, and, in the case of these ideas, access to tools to provide objective data to make meaningful lifestyle change. In a community where a person is a peer, the actions they take and learn from are that their volition and result in increased agency (or autonomy) or self-efficacy. This not only increase the chances of continuous lifestyle improvement, but improved outcomes throughout the heath system.

The health system and many lifestyle apps tell patients the “right thing to do”, which could be right, but given the complexity of lifestyle change is likely not to occur. Often, if the actions work, they can make the patient more dependent on things outside the patient's control. Communities share experiences, not expertise, which patients can try and, if they work for them, is a success. In some cases, success can range from slowing the progression of adverse health conditions to managing a disease, or the overall risk level in a population.

The use of the community (and the DCN and apps which connect patients to the community) can help overcome the above limitations. As one example, the community teaches and encourages members to discover their individual biological marker levels (such as for chronic systemic inflammation (CSI)) and, often more importantly, whether it is increasing or decreasing for the purpose of disease management. The Community encourages individuals to share what they are doing and how it impacted their condition and disease management. Finally, the members of the community can use these experiences to find things they can try and share based on the members having the same types of motivations for their actions and/or communications.

The DCN can also manage a member's communication feed to prioritize messages which contain ideas that might be applicable to them. This can be based on various factors, such as a motivation behind different messages or actions, a degree to which a message contains an idea, and a degree to which the idea will spread in the DCN.

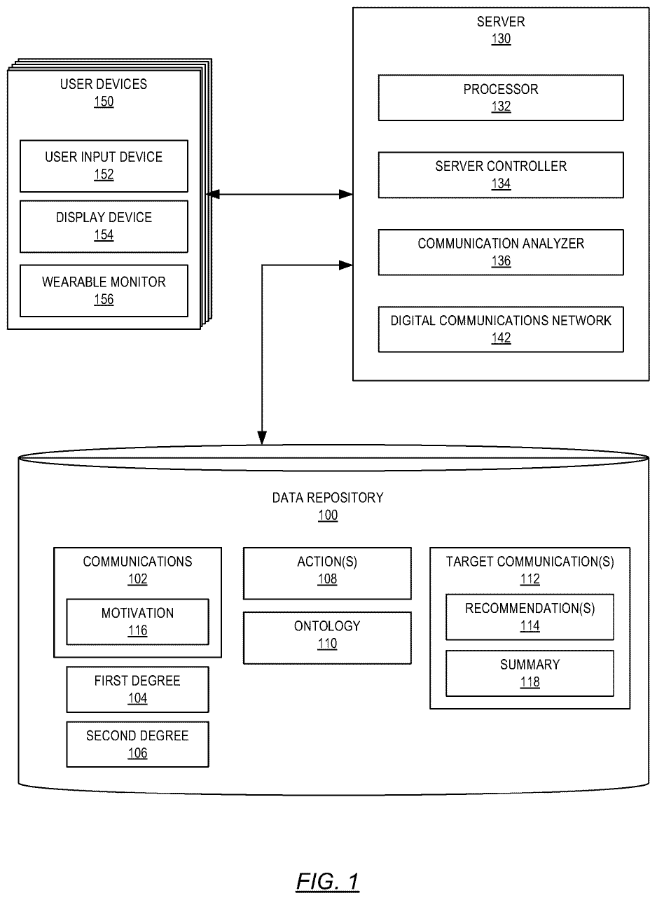

Turning to the Figures, the system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communications (102). The communications (102) are communications from members in a digital communication network (DCN) (142) (described in more detail below). The communications (102) may be, for example, text-based, image-based, multimedia communications, audio communications, or any combinations thereof. The communications may be between members, communications in a forum, communications with the DCN (142), or communications generated by the DCN (142) (e.g., communications from a conversation bot, also referred to as a chat bot). The communications (102) may include, for example, a post, a direct message, a comment, and/or visual and/or audio media by one or more members of the DCN (142) or generated by the DCN (142) (or a server controller such as a server controller (134) of the DCN (142)). The communications (102) may also be referred to as bot communications when the communications are generated and received from one or more conversation bots of the DCN (142).

The communications (102) in the DCN (142) include one or more motivations (116). The motivations (116) can include an idea useful in slowing a progression of an adverse health condition. The idea can include, for example actions (108) (described below) and/or an experience taken by the member who generated the communication (102). The idea can be obtained from or based on an ontology (110) (described below). The motivation can also relate to improving or reducing an adverse health condition experienced by the member who generated the communication (102).

The data repository (100) also stores a first degree (104). The first degree (104) is a degree to which a communication (102) contains an idea that is useful in slowing a progression of an adverse health condition. The idea may include an action (108) (described below) and/or an experience undertaken by the member who generated the communication. As similarly described above, the idea can be obtained from or based on an ontology (110) (described below).

The data repository (100) also stores a second degree (106). The second degree (106) is a degree to which the idea of the communication (102) will spread in the DCN (142). The second degree (106) may be based on, for example, a member's influence in the DCN (142). For example, communications from a member that can influence a majority of members of the DCN (142) will have a higher level of the second degree (106) than communications from a member who is only known to twenty or less members of the DCN (142).

The data repository (100) also stores action(s) (108). The actions (108) are actions that can be taken by the members of the DCN (142). The actions (108) can include lifestyle changes, medical interventions, changes in social interactions, and changes in a living environment. For example, the actions (108) can include yoga, a prescription, hiking, jogging, in-person meeting versus online meeting, etc.

The data repository (100) also stores ontologies (110). The ontology (110) is a defined set of relationships between the adverse health condition, ideas, motivations, and/or reflections and how the adverse health condition, ideas, motivations, and/or reflections relate to each other. The ontology (110) is used to, for example, define and show the interdependency of the individual member's adverse health condition and reflection.

In some embodiments, a language model such as a large language model can be used to develop the ontology (110). For example, the language model can be prompted with communications (102), the first degree (104), the second degree (106), and/or the motivation (116) of each communication (102) and instructions to develop the ontology (110). More specifically, the language model can determine relationships between communications (102) with similar a first degree (104), second degree (106), and/or motivation (116) or relationships between the first degree (104), second degree (106), and/or motivation (116) themselves.

The data repository (100) also stores target communication(s) (112). The target communications (112) are communications directed to an individual member of the plurality of members and are generated by the server controller (134) of the DCN (142) for the individual member. The target communication (112) may be generated based on an adverse health condition of the individual member, the first degree (104), the second degree (106), and/or the motivation (116). For example, the target communication (112) may include actions (108) taken by another member related to the improving the adverse health condition.

In some embodiments, the target communication (112) may include a recommendation (114). The recommendation (114) may include recommendations for the member to perform one or more of the set of actions (108). In another example, the recommendation (114) may recommend a tool for the member to use. The tool may be, for example, associated with living a healthy lifestyle and can include a wearable device configured to measure a biomarker, a kit to measure a biomarker, a health-inducing game, a psychometric instrument, or a health-improving program. Such tools can be used to track or monitor the member's health or an adverse health condition. Such tools can also be used to determine if actions (108) taken by the member have improved their health or adverse health condition.

In other embodiments, the target communication (112) may include a summary (118) of communications (102) from other members of the DCN (142). The summary (118) may describe a subset of communications (102) from a plurality of communications that are similar to each other. The subset of communications (102) may also have higher associated levels of the first degree (104) and/or the second degree (106).

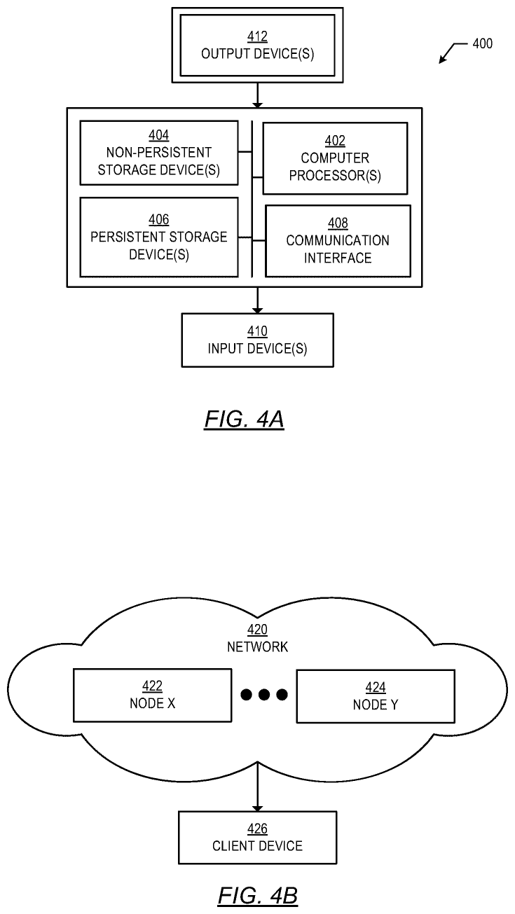

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136). An example of a computer system and network that may form the server (130) is described with respect to FIG. 4A and FIG. 4B.

The server (130) also includes a computer processor (132). The computer processor (132) is one or more hardware or virtual processors which may execute computer readable program code that defines one or more applications, such as the communication analyzer (136). An example of the computer processor (132) is described with respect to the computer processor(s) (402) of FIG. 4A.

The server (130) also may include a server controller (134). The server controller (134) is software or application specific hardware which, when executed by the computer processor (136), controls and coordinates operation of the software or application specific hardware described herein. Thus, the server controller (134) may control and coordinate execution of the communication analyzer (136).

The server (130) also includes the communication analyzer (136). The communication analyzer (136) is software or application specific hardware which, when executed by the computer processor (132), analyzes communication (102) in the DCN (142), determines the first degree (104), the second degree (106), and/or the motivation (116) of each communication (102) and generates the target communications (112). In other instances, the communication analyzer (136) analyzes communication (102) in the DCN (142), determines the first degree (104), the second degree (106), and/or the motivation (116) of each communication (102) based on the analysis, and prioritizes an order of communications (102) to present to an individual member.

The server (130) also includes a digital communications network (DCN) (142). The DCN (142) is a network through which members of a population can interact with each other, or with a system supported by the DCN (142). The DCN (142) can also interact with the members of the population and, for example, transmit communications (102) and target communications (112) to one or more members of the DCN (142).

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (400) shown in FIG. 4A) that communicate with the server (130). The user devices (150) may include a wearable monitor (156). In an alternative embodiment, a separate wearable device may be in communication with the user device (150), such as a smart watch, or blood pressure monitor. The user devices (150) may also include a user input device (152) and/or a display device (154).

While FIG. 1 shows a configuration of components, other configurations may be used without departing from the scope of one or more embodiments. For example, various components may be combined to create a single component. As another example, the functionality performed by a single component may be performed by two or more components.

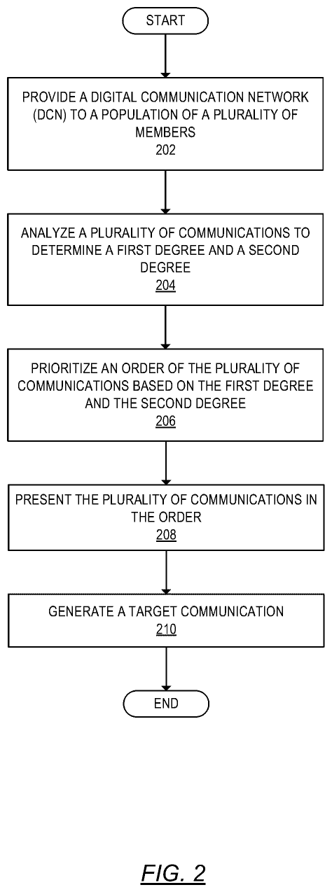

FIG. 2 is a logic flow diagram that illustrates a method, and a result of execution of computer program instructions, in accordance with various embodiments. The method can be used to prioritize an order of communications based on a first degree and a second degree of a communication. The method can also, in other instances, be used in part to facilitate or accomplish at least one of: manage the health care cost of the population; reduce the health care risk in the population; and/or slow the progression of an adverse health condition. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 202, a step of providing a digital communication network (DCN) to a population is provided. The DCN may be the same as or similar to the DCN (142). As previously described, members of the population enrolled in the DCN can communicate with each other or with the DCN itself. The members of the population can also communication with one or more conversation bots.

At Block 204, a step of analyzing a plurality of communications to determine a first degree and a second degree for each communication is provided. The plurality of communications includes communications such as the communications (102) and the first degree may be the same as or similar to the first degree (104) and the second degree may be the same as or similar to the second degree (106). As previously described, the first degree is a degree to which the communication contains an idea useful in slowing a progression of an adverse health condition and the second degree is a degree to which the idea will spread in the DCN. The idea can include an action or experience by the user who generated the communication and a motivation behind the action or experience. For example, the idea can include that the user or member added stretching to their daily routine with a motivation to alleviate chronic pain.

Analyzing the plurality of communications can include using the plurality of communications as input to a communication analyzer such as the communication analyzer (136) and receiving the first degree and the second degree. In other embodiments, a language model such as, for example, a large language model may be prompted with the plurality of communications and instructions to determine the first degree and the second degree of each communication. In such embodiments, the first degree and the second degree are received as output from the language model.

At Block 206, a step of prioritizing an order of the plurality of communications is provided. The order of the plurality of communications may be prioritized based on the first degree and the second degree. By prioritizing communications with higher first degrees and/or second degrees, an individual member may be more receptive to the prioritized communications that are related to the individual member's adverse health condition and are likely to appeal to a broader audience within the DCN, including the individual member.

In other embodiments, the order may be prioritized in which the individual member sees the plurality of messages based on a usefulness in slowing the progression of the adverse health condition. The usefulness may be determined by determining an adverse health condition of the individual member and prioritizing communications with the same adverse health condition as the individual member. For example, the target individual member may have chronic back pain and thus, communications directed to chronic back pain may be prioritized over communications directed to a broken bone.

At Block 208, a step of presenting the plurality of communications in the order to an individual member is provided. The plurality of communications may be presented to the individual member via a user interface such as the user interface (150). The plurality of communications may be presented one communication at a time, or presented all at once to the individual member. The plurality of communication may also be presented one communication at a time interval such as, for example, once every hour, every day, or every week.

At Block 210, a step of generating a target communication is provided. It will be appreciated that the Block 210 may be an optional step. The target communication may be the same as or similar to the target communication (112). The target communication can be based on the first degree or the second degree. The target communication may include some of the communications with high associated levels of the first degree and/or the second degree.

The method described in FIG. 2 can include more or less steps. One or more steps or any combination of steps may also be repeated in the method described in FIG. 2.

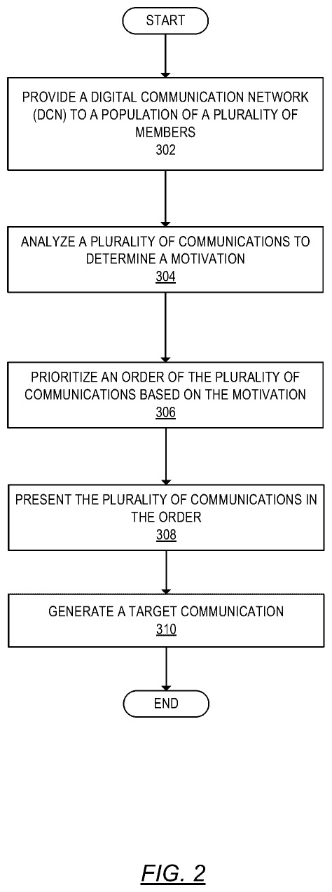

FIG. 3 is a logic flow diagram that illustrates a method, and a result of execution of computer program instructions, in accordance with various embodiments. The method can be used to prioritize an order of communications based on a motivation of a communication. The method can also, in other instances, be used in part to facilitate or accomplish at least one of: manage the health care cost of the population; reduce the health care risk in the population; and/or slow the progression of an adverse health condition. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 302, a step of providing a digital communication network (DCN) to a population is provided. The DCN may be the same as or similar to the DCN (142). As previously described, members of the population enrolled in the DCN can communicate with each other or with the DCN itself. The members of the population can also communication with one or more conversation bots.

At Block 304, a step of analyzing a plurality of communications to determine a motivation for each communication is provided. The plurality of communications includes communications such as the communications (103) and the motivation may be the same as or similar to the motivation (116). Analyzing the plurality of communications can include using the plurality of communications as input to a communication analyzer such as the communication analyzer (136) and receiving the motivation. In other embodiments, a language model such as, for example, a large language model may be prompted with the plurality of communications and instructions to determine the motivation of each communication. In such embodiments, the motivation is received as output from the language model.

The motivation may correlate to the members' motivation for interacting with the idea. For example, the spread of the idea that wearable device such as a continuous glucose monitor can provide information and understanding of a member's lifestyle is mediated by whether the individual member seeing the idea is motivated by disease management or weight loss or social position in the network. Thus, the motivation is as important in understanding contagion as attributes of the idea itself.

At Block 306, a step of prioritizing an order of the plurality of communications is provided. The order of the plurality of communications may be prioritized based on the motivation. By prioritizing communications with motivations similar to a motivation of an individual member, the individual member may be more receptive to the prioritized communications that are related to the individual member's adverse health condition and motivation.

Prioritizing the order in which the individual member sees the plurality of messages may based on a usefulness in slowing the progression of the adverse health condition. The usefulness may be determined by determining an adverse health condition of the individual member and prioritizing communications with the same adverse health condition as the individual member. For example, the individual member may have chronic back pain and thus, communications directed to chronic back pain may be prioritized over communications directed to a broken bone. Communications with the same or similar motivations as the user may also be prioritized. For example, the individual member may be motivated to manage their chronic back pain and communications that are also motivated by managing chronic pain may be prioritized over communications that are motivated by socialization.

At Block 308, a step of presenting the plurality of communications in the order to an individual member is provided. The plurality of communications may be presented to the individual member via a user interface such as the user interface (150). The plurality of communications may be presented one communication at a time, or presented all at once to the individual member. The plurality of communication may also be presented one communication at a time interval such as, for example, once every hour, every day, or every week.

At Block 310, a step of generating a target communication is provided. It will be appreciated that the Block 310 may be an optional step. The target communication may be the same as or similar to the target communication (112). The target communication can be based on the motivation. The target communication may include some of the communications with motivation(s) that are the same as or similar to motivations of the individual member.

The method described in FIG. 3 can include more or less steps. One or more steps or any combination of steps may also be repeated in the method described in FIG. 3.

The various blocks shown in FIGS. 2-3 may be viewed as method steps, as operations that result from use of computer program code, and/or as one or more logic circuit elements constructed to carry out the associated function(s). The methods described in FIGS. 2-3 may be used to manage a disease, lower a health risk in a population, manage health care costs, and increase or improve the effectiveness of communications in the DCN.

In other embodiments of any one of the methods above, the idea contained in each communication is determined automatically. The idea can also include at least one of an action or an experience undertaken by the member who generated the communication. The idea can include a motivation of the action. Similarly, the adverse condition for each communication can be determined automatically. Further, the adverse condition associated with the member for who the communications are prioritized for can also be determined automatically.

In additional embodiments of any one of the methods above, the method also includes analyzing communications in the DCN to generate an ontology of health conditions for the individual. More specifically, the idea for each communication can be associated with an ontology. Similarly, the adverse condition for each communication can also be associated with an ontology. Further, the adverse condition associate with the member for who the communications are prioritized for can also be associated with an ontology.

The ontology of health conditions includes the adverse health condition. An ontology is a way of categorizing the properties of a subject area and describe how they are related. The ontology can define a set of terms and relational expressions that represent the entities in that subject area. The ontology defines the concept behind the language uses and may be associated with various terms and phrases. The DCN is able to analyze the words used by the members in order to determine the underlying idea, for example, exhaustion may be associated with feelings of tiredness, lack of sleep, etc.

In embodiments of any one of the methods above, the DCN can include an AI bot configured to generate messages based on an analysis of communication in the DCN or from biometrics provided to the DCN. The biometrics may be derived from a wearable device, a biometric sample, or answers to a survey or psychometric instrument.

The AI bot may include a language model such as a large language model (LLM), for example, configured to generate text. The AI bot may also include a visual language model that can generate text based on visual content. In any embodiment, the AI bot can receive different types of input (e.g., text, images video) and generate different types of output (e.g., text, images video). The AI bot may be configured to automatically generate the messages based, at least in part, on analysis of communications involving the individual on the DCN. The AI bot may also be configured to generate the messages based, at least in part, on previous conversations on the DCN between the individual and the AI bot.

In additional embodiments of any one of the methods above, the method also includes generating a paraphrased communication of multiple communications and presenting the paraphrased communication to the individual member. The paraphrased communication may be generated by, for example, the language model.

In other embodiments of any one of the methods above, the adverse health condition may include at least one of: chronic systemic inflammation, malaise, and low energy.

In further embodiments of any one of the methods above, the adverse health condition may be a disease. The disease may be obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and/or anxiety.

In additional embodiments of any one of the methods above, the adverse health condition may be a prodromal version of the disease.

In other embodiments of any one of the methods above, the adverse health condition may be a health risk. The health risk may be one of: a risk of falling, and a risk of becoming infected.

In further embodiments of any one of the methods above, the adverse health condition may be social dysfunction.

In additional embodiments of any one of the methods above, communications in the DCN may include at least one of: one-to-one communications, one-to-many communications, one-to-system communications, system-to-one communications, and system-to-many communications.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 4A, the computing system (400) may include one or more computer processor(s) (402), non-persistent storage device(s) (404), persistent storage device(s) (406), a communication interface (408) (e.g., Bluetooth interface, infrared interface, network interface, optical interface, etc.), and numerous other elements and functionalities that implement the features and elements of the disclosure. The computer processor(s) (402) may be an integrated circuit for processing instructions. The computer processor(s) (402) may be one or more cores, or micro-cores, of a processor. The computer processor(s) (402) includes one or more processors. The computer processor(s) (402) may include a central processing unit (CPU), a graphics processing unit (GPU), a tensor processing unit (TPU), combinations thereof, etc.

The input device(s) (410) may include a touchscreen, keyboard, mouse, microphone, touchpad, electronic pen, or any other type of input device. The input device(s) (410) may receive inputs from a user that are responsive to data and messages presented by the output device(s) (412). The inputs may include text input, audio input, video input, etc., which may be processed and transmitted by the computing system (400) in accordance with one or more embodiments. The communication interface (408) may include an integrated circuit for connecting the computing system (400) to a network (not shown) (e.g., a local area network (LAN), a wide area network (WAN) such as the Internet, mobile network, or any other type of network) or to another device, such as another computing device, and combinations thereof.

Further, the output device(s) (412) may include a display device, a printer, external storage, or any other output device. One or more of the output device(s) (412) may be the same or different from the input device(s) (410). The input device(s) (410) and output device(s) (412) may be locally or remotely connected to the computer processor(s) (402). Many different types of computing systems exist, and the aforementioned input device(s) (410) and output device(s) (412) may take other forms. The output device(s) (412) may display data and messages that are transmitted and received by the computing system (400). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (402), is configured to perform one or more embodiments, which may include transmitting, receiving, presenting, and displaying data and messages described in the other figures of the disclosure.

The computing system (400) in FIG. 4A may be connected to, or be a part of, a network. For example, as shown in FIG. 4B, the network (420) may include multiple nodes (e.g., node X (422) and node Y (424), as well as extant intervening nodes between node X (422) and node Y (424)). Each node may correspond to a computing system, such as the computing system shown in FIG. 4A, or a group of nodes combined may correspond to the computing system shown in FIG. 4A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (400) may be located at a remote location and connected to the other elements over a network.

The nodes (e.g., node X (422) and node Y (424)) in the network (420) may be configured to provide services for a client device (426). The services may include receiving requests and transmitting responses to the client device (426). For example, the nodes may be part of a cloud computing system. The client device (426) may be a computing system, such as the computing system shown in FIG. 4A. Further, the client device (426) may include or perform all or a portion of one or more embodiments.

The computing system of FIG. 4A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

The present invention may be further exemplified by one, or a combination of one or more of, the following statements:
Statement 1. A method to slow a progression of an adverse health condition in an individual member of a population, the method comprising: providing a digital communication network (DCN) for a plurality of members of the population to communicate with each other; analyzing a plurality of communications on the DCN, the plurality of communications generated by the plurality of members; determining, based on the analysis of the plurality of communications, a first degree to which each communication contains an idea that is useful in slowing a progression of an adverse health condition and a second degree to which an idea will spread in the DCN; prioritizing an order in which the individual member sees the plurality of communications based on at least the first degree to which the communication contains the idea that is useful in slowing the progression of the adverse health condition that the member has and the second degree to which the idea will spread in the DCN; and presenting the plurality of communications to the individual member in the order, wherein messages with higher levels of prioritization are shown first.
Statement 2. The method of Statement 1, wherein the idea contained in the communication is determined automatically.
Statement 3. The method of Statement 2, wherein the idea includes at least one of an action and an experience undertaken by the member who generated the communication, and wherein the idea includes a motivation of the action by the member who generated the communication.
Statement 4. The method of Statement 3, wherein the idea is associated with an ontology created automatically based on the analysis of the plurality of communications.
Statement 5. The method of Statement 2, wherein the analysis of the plurality of communications to determine the idea is executed by a language model.
Statement 6. The method of Statement 6, wherein the language model comprises a large language model.
Statement 7. The method of Statement 1, wherein the adverse health condition contained in the communication is determined automatically.
Statement 8. The method of Statement 7, wherein the adverse health condition is associated with an ontology created automatically based on the analysis of the plurality of communications.
Statement 9. The method of Statement 7, wherein the analysis of the plurality of communications to determine the adverse health condition is executed by a language model
Statement 10. The method of Statement 9, wherein the language model comprises a large language model.
Statement 11. The method of Statement 1, wherein the adverse health condition associated with the individual member is determined by automatically analyzing one or more communications of the individual member.
Statement 12. The method of Statement 11, wherein the adverse health condition associated with the individual member is associated with an ontology created automatically based on the analysis of the plurality of communications.
Statement 13. The method of Statement 11, wherein the analysis of the one or more communications of the individual member is executed by a language model.
Statement 14. The method of Statement 13, wherein the language model comprises a large language model.
Statement 15. The method of Statement 1, wherein the adverse health condition comprises chronic systemic inflammation, malaise, low energy, a disease, a health risk, and a social dysfunction.
Statement 16. The method of Statement 15, wherein the adverse health condition comprises a disease and the disease is at least one of obesity, diabetes, rheumatoid arthritis, Crohn's disease, psoriasis, eczema, cardiovascular disease, congestive heart failure, chronic obstructive pulmonary disease, insomnia, sleep quality disorders, asthma, depression, and anxiety.
Statement 17. The method of Statement 15, wherein the disease is at least one of a prodromal disease or prodromal condition.
Statement 18. The method of Statement 15, wherein the condition comprises the health risk and the health risk comprises the individual member falling and the individual member becoming infected.
Statement 19. The method of Statement 1, further comprising: generating, using a language model, a paraphrased communication of the plurality of communications presented to the individual member; and presenting the paraphrased communication to the individual member.
Statement 20. The method of Statement 1, wherein the communication between the plurality of members is one to one, one to many, one to system, system to one, system to many.
Statement 21. The method of Statement 20, wherein the system is an artificial intelligence bot that derives its communications from the analysis of the communications in the digital communication network or biometrics provided to the system.
Statement 22. The method of Statement 21, wherein the biometric is derived from at least one of: a wearable device, a biometric sample, and answers to a survey or psychometric instrument.
Statement 23. A method to slow progression of an adverse health condition in an individual member of a population, the method comprising: providing a digital communication network (DCN) for a plurality of members of the population to communicate with each other; analyzing a plurality of communications on the DCN, the plurality of communications generated by the plurality of members; determining, based on the analysis of the plurality of communications, a motivation for each communication of the plurality of communications that contains an idea useful in slowing a progression of an adverse health condition; prioritizing an order in which the individual member sees the plurality of communications based on the motivation of each communication of the plurality of communications; and presenting the plurality of communications to the individual member in the order, wherein communications with motivations matching a motivation of the individual member are prioritized higher.
Statement 24. The method of Statement 23, wherein the motivation of each communication of the plurality of communications is determined automatically.
Statement 25. The method of Statement 24, wherein the analysis of the plurality of communications to determine the motivation is executed by a language model.
Statement 26. The method of Statement 25, wherein the language model comprises a large language model.
Statement 27. The method of Statement 23, wherein the idea contained in the communication is determined automatically.
Statement 28. The method of Statement 27, wherein the idea includes at least one of an action and an experience undertaken by the member who generated the communication, and wherein the idea includes a motivation of the action by the member who generated the communication.
Statement 29. The method of Statement 28, wherein the idea is associated with an ontology created automatically based on the analysis of the plurality of communications.
Statement 30. The method of Statement 23, wherein the analysis of the plurality of communications to determine the idea is executed by a language model.
Statement 31. The method of Statement 30, wherein the language model comprises a large language model.
Statement 32. The method of Statement 23, wherein the adverse health condition contained in the communication is determined automatically.
Statement 33. The method of Statement 32, wherein the adverse health condition is associated with an ontology created automatically based on the analysis of the plurality of communications.
Statement 34. The method of Statement 32, wherein the analysis of the plurality of communications to determine the adverse health condition is executed by a language model.
Statement 35. The method of Statement 34, wherein the language model comprises a large language model.
Statement 36. The method of Statement 23, wherein the adverse health condition associated with the individual member is determined by automatically analyzing one or more communications of the individual member.
Statement 37. The method of Statement 36, wherein the adverse health condition associated with the individual member is associated with an ontology created automatically based on the analysis of the plurality of communications.
Statement 38. The method of Statement 36, wherein the analysis of the one or more communications of the individual member is executed by a language model.
Statement 39. The method of Statement 38, wherein the language model comprises a large language model.
Statement 40. The method of Statement 23, wherein the adverse health condition comprises chronic systemic inflammation, malaise, low energy, a disease, a health risk, and a social dysfunction.
Statement 41. The method of Statement 40, wherein the adverse health condition comprises a disease and the disease is at least one of obesity, diabetes, rheumatoid arthritis, Crohn's disease, psoriasis, eczema, cardiovascular disease, congestive heart failure, chronic obstructive pulmonary disease, insomnia, sleep quality disorders, asthma, depression, and anxiety.
Statement 42. The method of Statement 41, wherein the adverse disease is prodromal.
Statement 43. The method of Statement 41, wherein the condition comprises the health risk and the health risk comprises the individual member falling and the individual member becoming infected.
Statement 44. The method of Statement 23, further comprising: generating, using a language model, a paraphrased communication of the plurality of communications presented to the individual member; and presenting the paraphrased communication to the individual member.
Statement 45. The method of Statement 23, wherein the communication between the plurality of members is one to one, one to many, one to system, system to one, system to many.
Statement 46. The method of Statement 45, wherein the system is an artificial intelligence bot that derives its communications from the analysis of the communications in the digital communication network or biometrics provided to the system.
Statement 47. The method of Statement 46, wherein the biometric is derived from at least one of: a wearable device, a biometric sample, and answers to a survey or psychometric instrument.
Statement 48. The method of Statement 1, wherein the method is used to reduce health care costs in the population.
Statement 49. The method of Statement 1, wherein the method is used to reduce health care risk in the population.
Statement 50. The method of Statement 1, wherein the method is used to manage a disease in at least one of the population and one or more members of the population.
Statement 51. The method of Statement 23, wherein the method is used to reduce health care costs in the population.
Statement 52. The method of Statement 23, wherein the method is used to reduce health care risk in the population.
Statement 53. The method of Statement 23, wherein the method is used to manage a disease in at least one of the population and one or more members of the population.

What is claimed is:

1. A method comprising:
providing a digital communication network (DCN) for a plurality of members of the population to communicate with each other;
analyzing a plurality of communications on the DCN, the plurality of communications generated by the plurality of members;
determining, based on the analysis of the plurality of communications, a first degree to which each communication contains an idea that is useful in slowing a progression of an adverse health condition and a second degree to which an idea will spread in the DCN;
prioritizing an order in which the individual member sees the plurality of communications based on at least the first degree to which the communication contains the idea that is useful in slowing the progression of the adverse health condition that the member has and the second degree to which the idea will spread in the DCN; and
presenting the plurality of communications to the individual member in the order, wherein messages with higher levels of prioritization are shown first.

2. The method of claim 1, wherein the idea includes at least one of an action and an experience undertaken by the member who generated the communication, and wherein the idea includes a motivation of the action by the member who generated the communication.

3. The method of claim 2, wherein the action comprises at least one of: lifestyle changes, medical interventions, changes in social interactions, and changes in a living environment.

4. The method of claim 1, wherein the idea is associated with an ontology created automatically based on the analysis of the plurality of communications.

5. The method of claim 1, wherein the adverse health condition is associated with an ontology created automatically based on the analysis of the plurality of communications.

6. The method of claim 1, wherein determining the first degree and the second degree includes prompting a language model with the plurality of communications and instructions to determine the first degree and the second degree.

7. The method of claim 6, wherein the language model is a large language model.

8. The method of claim 1, wherein the individual member has the adverse health condition.

9. The method of claim 8, wherein the method is used to slow the progression of the adverse health condition of the individual member.

10. The method of claim 9, wherein the adverse health condition is determined by automatically analyzing communications from the individual member in the DCN.

11. The method of claim 10, wherein the adverse health condition comprises at least one of chronic systemic inflammation, malaise, low energy, a disease, a health risk, social dysfunction, or a prodromal disease.

12. The method of claim 11, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

13. The method of claim 11, wherein the health risk is at least one of falling or becoming infected with an illness.

14. A method comprising:
providing a digital communication network (DCN) for a plurality of members of the population to communicate with each other;
analyzing a plurality of communications on the DCN, the plurality of communications generated by the plurality of members;
determining, based on the analysis of the plurality of communications, a motivation for each communication of the plurality of communications that contains an idea useful in slowing a progression of an adverse health condition;
prioritizing an order in which the individual member sees the plurality of communications based on the motivation of each communication of the plurality of communications; and
presenting the plurality of communications to the individual member in the order, wherein communications with motivations matching a motivation of the individual member are prioritized higher.

15. The method of claim 14, wherein the idea includes at least one of an action and an experience undertaken by the member who generated the communication, and wherein the idea includes a motivation of the action by the member who generated the communication.

16. The method of claim 15, wherein the action comprises at least one of: lifestyle changes, medical interventions, changes in social interactions, and changes in a living environment.

17. The method of claim 14, wherein the idea is associated with an ontology created automatically based on the analysis of the plurality of communications.

18. The method of claim 17, wherein the adverse health condition comprises at least one of chronic systemic inflammation, malaise, low energy, a disease, a health risk, social dysfunction, or a prodromal disease.

19. The method of claim 14, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

20. A system comprising:
a computer processor;
a data repository in communication with the computer processor and storing:
communications,
a first degree, and
a second degree;
a digital communications network (DCN) which, when executed by the computer processor, provides a network for a plurality of members of a population to interact with at least each other or the DCN; and
a server controller which, when executed by the computer processor:
analyzes communications on the DCN, the communications generated by the plurality of members;
determines, based on the analysis of the communications, a first degree to which each communication contains an idea that is useful in slowing a progression of an adverse health condition and a second degree to which an idea will spread in the DCN;
prioritizes an order in which the individual member sees the communications based on at least the first degree to which the communication contains the idea that is useful in slowing the progression of the adverse health condition that the member has and the second degree to which the idea will spread in the DCN; and
presenting the communications to the individual member in the order, wherein messages with higher levels of prioritization are shown first.
